(19) United States
(12) Patent Application Publication
(10) Pub. No.: US 2018/0008449 A1
(43) Pub. Date: Jan. 11, 2018

(54) Self-Fitting, Self-Adjusting, Automatically Adjusting and/or Automatically Fitting Orthopedic or other (e.g. non human use) Immobilization Splint or Device

(71) Applicant: Peter A. Feinstein, Shavertown, PA (US)

(72) Inventor: Peter A. Feinstein, Shavertown, PA (US)

(21) Appl. No.: 15/203,536

(22) Filed: Jul. 6, 2016

(51) Int. Cl. A61F 5/058; A44B 11/25; A41F 1/00; A41F 1/04; A61F 5/03; A44B 18/00

(52) U.S. Cl. CPC A61F 5/058; A61F 5/05858; A61F 5/03; A44B 18/00; A41F 1/00; A41F 1/04; A41F 1/002; A44B 11/25

(57) ABSTRACT

Provided is a self-fitting and automatically adjustable clasp band/strap which is usable in medical immobilization (e.g., casts, splints, braces) and multiple other fixation devices. The clasp band/strap may have a shape memory material and clasp members attached to two ends of the clasp band/strap. Upon stimulation by a trigger source, the shape memory material deforms which brings the two end portions closer to each other, causing the two clasp members to attach to each other to form a closure. Additionally, the clasp band/strap may include a motor, a control unit, and sensors to enable a motor actuated fine tensioning. Finally, the clasp band/strap may have an adhesive backing or any other kind of annealing or connective backing, such as a Velcro strap with an adhesive backing, for attaching to a splint or other object.

### FIELD OF THE INVENTION

The invention relates generally to immobilization devices that are useful in the orthopedics field. More particularly, the invention relates to immobilization devices with self-fitting, self-adjusting, automatically adjusting and/or automatically fitting ability.

### BACKGROUND OF THE INVENTION

Medical immobilization and/or fixation devices (e.g., casts, splints, braces) are commonly used to heal broken bones, tendon tears, or other injuries of a subject's limbs. Conventional means to tighten, fasten or close casts, splints, or braces often require a user to use both hands to secure the casts, splints, or braces about a limb. For example, Velcro™ straps and buckles require a user to grasp one end of a strap with one hand while holding the other end of the strap and the brace in position with the other hand in order to fasten the strap of the brace. Proper fitting of such braces may be difficult and/or challenging. One example would be the case of orthopedic immobilization for a patient, especially when the patient is dexterity challenged or the brace is being fit to the arm or hand.

Shape memory materials, such as shape memory polymers (SMP) and shape memory alloys (SMA) have been used in medical immobilization and fixation devices in recent years. Both SMPs and SMAs have the ability to return from a deformed state (temporary shape) to an original (e.g., baseline, memorized, permanent) shape induced by an external stimulus. For example, an SMP can exhibit change from a rigid state to an elastic state, then back to the rigid state using an external stimulus. The SMP in the elastic state can recover its “permanent” shape if left unrestrained. In similar respects, an SMA is an alloy that remembers its original shape and after undergoing deformation, is able to transform back to its pre-deformed, original shape when triggered to do so. As such, shape memory materials can be useful in various applications ranging from, for example, medical immobilization and/or fixation devices (e.g., casts, splints, braces).

U.S. Pat. No. 5,607,756 describes a splint for practicing a method of correction on a foot. The splint comprises shape memory alloy wires, preferably in the form of either woven fabric, such as a mesh, or a nonwoven fabric plate. The shape memory alloy wires preferably consist of a Ti—Ni series alloy exhibiting superelasticity at a normal or used temperature. For use, the splint is first put inside a shoe and the splint recovers its original shape at a predetermined internal temperature.

U.S. Pat. No. 8,100,843 reports a medical cast for an injured limb of a subject. The medical cast comprises a SMP which is interchangeable from a temporary shape to a permanent shape upon heating. By the shape transition of the SMP, the cast is able to conform to the shape of the injured limb.

US 2013/0303957 discloses body support bandages and orthoses for the human or animal body which have at least one element for providing body support and another element comprised of shape memory material for compression or introduction of pressure.

US 2014/0257156 discloses a medical brace embedded with nitinol wires. When activated by electricity, the nitinol wires deform, which causes the medical brace to shrink and to apply a local pressure to the body part. The brace is thus closed and tightened about a limb. US 2014/0257156 also discloses that the brace may include a motorized closure device for automatically opening, closing, and tightening the brace about a limb.

JP 2003144473 discloses a reusable splint stably attached to a fixing part of a lesion. This splint comprises shape memory resin sheets which, when heated to a glass transition temperature (Tg) or more, would mold along the shape of the fixing part of the lesion.

A drawback present in these applications is that the shape memory material has been pre-fixed in the supporting (main) part of splints, casts, or braces. Because each limb has different dimension, size, and contour, the casts, splints, or braces fashioned with pre-fixed SMP/SMA may not provide the best fit even after the initial SMP/SMA activation. Further, the shape memory materials which rely on the chemical characteristics of the particular SMP/SMA give one or two different end shape results/permutations, with no gradual or intermediate shapes based on feedback. But sometimes, after initial setting of the shape memory material, the limb underlying the shape memory material may slightly expand and/or contract as a result of healing (e.g., swelling dissipates, bone alignment improves, etc.). The SMP/SMA containing casts, splints, or braces may cease to be fitted accurately on the limb and/or correspond in shape to the limb.

Therefore, it would be beneficial to provide an immobilization and/or fixation orthopedic device or splint which provides self-assembling and self-closure around a limb without manually maneuvering of the device relative to the limb so that it is suitable for one handed or even hands free operation. Desirably, the immobilization and/or fixation orthopedic device or splint may also conform to the shape of a limb upon contact with the limb to provide a tight and directed fitting. It would also be desirable for the immobilization and/or fixation device to be able to automatically adjust the tightness and fitting after the initial contact and also during a course of treatment.

### SUMMARY OF THE INVENTION

It is an objective of the present invention to provide an immobilization and/or fixation device for orthopedic treatment which provides self-assembling and automatic closure around a limb without manually maneuvering of the device or item relative to the limb so that it is suitable for one handed or even hands free operation.

It is another objective of the present invention to provide an immobilization and/or fixation device for orthopedic treatment which may conform to the shape of a limb upon contact with the limb, and which may further automatically adjust the tension between the limb and the device to provide a desired fitting (a hands free operation).

It is a further objective of the present invention to provide an immobilization and/or fixation device for orthopedic treatment that is able to automatically adjust the tightness and fitting after the initial contact and also during a course of the treatment.

The present invention achieves these objectives by providing clasp bands/straps with self-fitting, self-adjusting, automatically adjusting and/or automatically fitting ability, which are particularly suitable for facilitating an automatic closure of or around a limb. The clasp bands/straps may be elongated bands/straps comprising a fabric layer on which a shape memory material and a non-shape memory material are deposited. The two ends of each of the clasp bands/straps may comprise two clasp members. The two clasp members are separated from one another in an open position and connect to each other in a closed position so as to connect the two ends of the clasp bands/straps.

The clasp bands/straps comprise a trigger source to provide a stimulus to the shape memory material which leads the clasp bands/straps to deform and bring the two ends of the clasp bands/straps to move toward each other around a limb. As the two ends move closer to each other, the two clasp members clasp to form a loop. In some embodiments, only one end of each of the clasp bands/straps receives a clasp member, while the other end is attached to an immobilization or fixation device (e.g. splint, cast, or brace). In this case, it requires two (“half”) clasp bands/straps to form a clasp. Both half bands/straps and one-piece bands/straps facilitate the closure by using the same shape memory material triggered clasp closure mechanism. The clasp bands/straps may have a Velcro strap backing for attaching to a corresponding Velcro strap on the surface of another immobilization or fixation device (e.g. splint, cast, or brace).

In a preferred embodiment, the stimulus is application of electric current. In another preferred embodiment, the clasp is a magnetic clasp. Thus the two clasp members include two magnetic pieces. The magnetic clasp may comprise a magnet shield on certain surfaces or parts of the clasp members to insulate the areas outside the magnetic pieces from magnetic force. A closed magnetic clasp may have a tab, an indentation, or a button on an edge of the clasp members so that a user may easily lift up or push away one of the clasp members with a finger in order to open the engaged clasp members. There may also be a tab, indentation, or button present for manual operation of loosening and tightening of the band/strap as an alternative to sensor feedback and control.

The clasp bands/straps may further comprise a motor disposed on one of the clasp members, one or more sensors disposed on the clasp bands/straps or on an object to which attach the clasp bands/straps, and a control unit. The sensors acquire information related to the clasp bands/straps (or the object) and the limb, and send sensed information to the control unit. The control unit then triggers the activation of the motor based on the sensed information. The movement of the motor adjusts the relative position of the clasp member with respect to the clasp band/strap. This is also called motor actuated fine tuning/tensioning.

The motor used in the adjustable clasp may be a worm-gear motor, a lead screw actuator, or a rack and pinion motor or any other motor assembly; the sensors may be touch sensors, pressure sensors, force sensors, capacitive sensors, conductivity sensors, light or optical sensors, heat sensors, strain gauges, stress gauges, bend sensors, magnetic sensors, location sensors, accelerometer sensors, mechanical sensors (e.g., external buttons or levels, removable tabs/rods/latches, external sliders, bending-release latches, etc.), or a combination thereof or any additional type of sensor A user may provide instructions related to the operation of the clasp hands to the control unit via a user input unit.

According to another embodiment, the present invention provides an immobilization and fixation device (e.g. a brace, a splint, a cast) which comprises a composite adapted to be placed around a body part and provide strength and weight-bearing support to the body part when it is in a closed, working position. The device may also include a plurality of the clasp bands/straps as described above for putting the composite in a closed position for orthopedics treatment. For example, the composite may include at least one foam layer to provide protection and comfort to the wearer and a fabric liner for contact with a body part. It may be a laminate or “stack up” composite with layers of foam/fabrics/actuators/circuitry/spacer/stiffeners. The plurality of clasp bands/straps may be permanently attached to the device composite by being sewn or otherwise permanently bonded to the device. Alternatively, the clasp bands/straps may be removably attached to the device by attaching to anchors, such as buckles, Velcro strap, or other adhesives that are on the device. Preferably, both the clasp bands/straps and the device use Velcro straps for attachment.

For use as part of a splint, the splint is first placed onto a body part. A trigger source is activated to provide a stimulus to the shape memory material, causing it to transform to a different form. The phase transformation further causes the clasp bands/straps to bend such that two distal end portions of the clasp bands/straps move toward each other (“self-assembly”). As the two end portions move closer to each other, two clasp members positioned on the two distal end portions of the bands/straps clasp to close the loop, without using a hand to manually pull a strap and fasten it onto a splint.

The immobilization and fixation device may also include components (e.g., a motor, sensors, a control unit, and a power source of any kind) to enable motor actuated fine tuning/tensioning. In the device, the sensors may be disposed on the inner layer of the composite for measurement and the motor may be placed in the composite to directly adjust its tightness. The power source may be internal or external to the device. Additionally, more than one motor and more than one controller may be used for individual control the fitting of the composite and the clasp bands/straps.

In some embodiments, the composite itself may comprise a shape memory material such that it may self assemble around a subject. Such self-assembly may trigger the clasp of a pair of clasp members if the clasp members are attached to the composite. This type of composite may be used to provide tight fitting garments, such as a corset or a waist belt with a standard buckle.

### BRIEF DESCRIPTION OF THE DRAWINGS

FIGS. 1A and 1B show an enlarged cross-sectional view and an isometric view of an embodiment of a clasp band/strap with parts removed to show internal details, in a disconnected position; FIG. 1C shows an enlarged cross-sectional view and an isometric view of an embodiment of a clasp band/strap with parts removed to show internal details, in a connected position.

FIG. 2A show an enlarged cross-sectional view and an isometric view of another embodiment of a clasp band/strap with parts removed to show internal details, in an open position; FIG. 2B shows an enlarged cross-sectional view and an isometric view of an embodiment of a clasp band/strap with parts removed to show internal details, in a looped position.

FIGS. 3A and 3B show an isometric view of an embodiment of a human orthopedic immobilization and fixation device in its open and closed positions.

FIGS. 4A-4C are step views of a material having self-assembly and adaptive shape adjustment capability undergoing self-assembly around an underlying object and thereafter disassembly from the underlying object.

FIG. 5A shows an isometric view of an embodiment of backing of a clasp band/strap. FIG. 5B shows an isometric view of a few embodiments of anchors of an immobilization and fixation device.

FIG. 6 shows a schematic view of an embodiment having a different mechanism to activate a motor.

FIG. 7 shows a schematic view of an embodiment having a different mechanism to stimulate a shape memory material.

FIG. 8 shows an isometric view of another embodiment of an immobilization and fixation device in its open and closed positions.

FIGS. 9A and 9B shows an isometric view of a process of applying the immobilization and fixation device for treating a body part of a patient.

FIGS. 10A to 10B are step views of a personalized and adjustable fitting garment being worn on a body part.

FIG. 11 shows an isometric view of an embodiment of an automatically connecting and self-fitting belt to be worn with pants around a waist of a person.

### DETAILED DESCRIPTION OF THE INVENTION

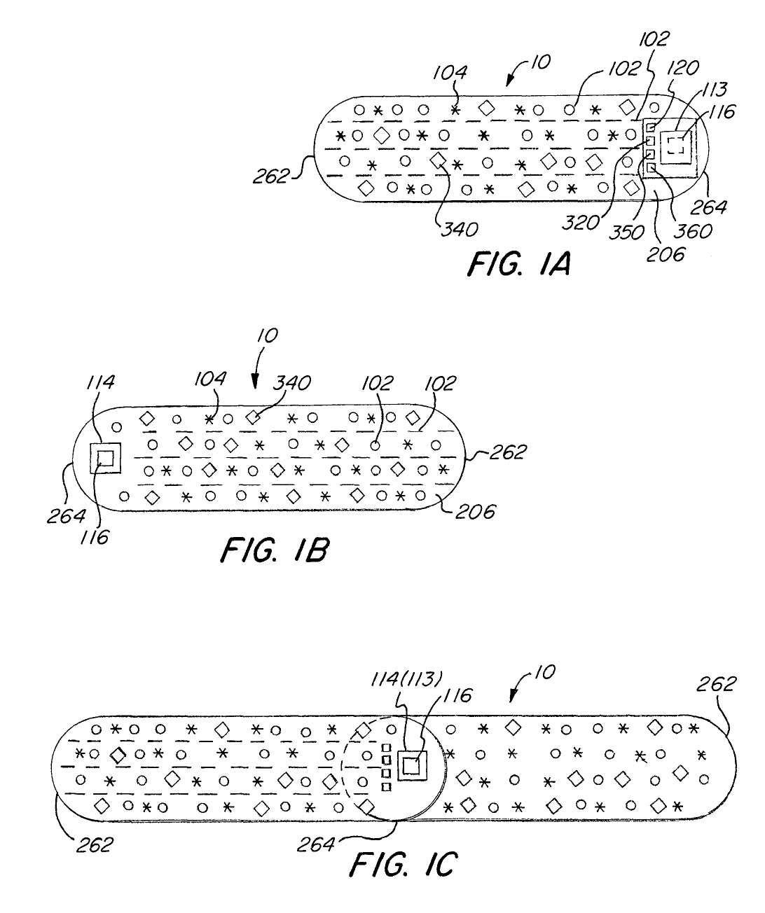

In one aspect, the present invention provides a clasp band/strap which has an automatic closure function which may be used to tighten casts, splints, and braces. As shown in FIGS. 1A and 1B, the clasp bands/straps 10 have elongated bands/straps. Though the bands/straps as shown have substantially the same width, such consistency in width is not required for the functions of the clasp bands/straps. The clasp bands/straps 10 comprise a shape memory material 102 and a non-shape memory material 104. The clasp bands/straps 10 may further comprise a liner 206 on which the shape memory material 102 and the non-shape memory material 104 are deposited. The clasp bands/straps 10 may comprise a trigger source 120 in communication with the shape memory material 102 and configured to provide a stimulus to the shape memory material 102.

The phrase “in communication with” with respect to the trigger source can mean that the trigger source has an effect, provides an effect, produces an effect on, and/or induces an effect on the shape memory material (e.g., transmit electricity to the shape memory material, pass a liquid to the shape memory material; transmit heat/cooling to the shape memory material; irradiate the shape memory material; adjust pH of shape memory material; effect a chemical reaction in the shape memory material, etc.). A preferred stimulus is application of electric current.

Each of the clasp bands/straps 10 has a proximal end 262 and a distal end 264. A clasp having two clasp members is provided for a pair of the clasp bands/straps. FIGS. 1A and 1B show that the clasp members 113, 114 are attached to the distal ends 264 of the pair of clasp bands/straps 10 so that the clasp may connect or disconnect the pair of clasp bands/straps.

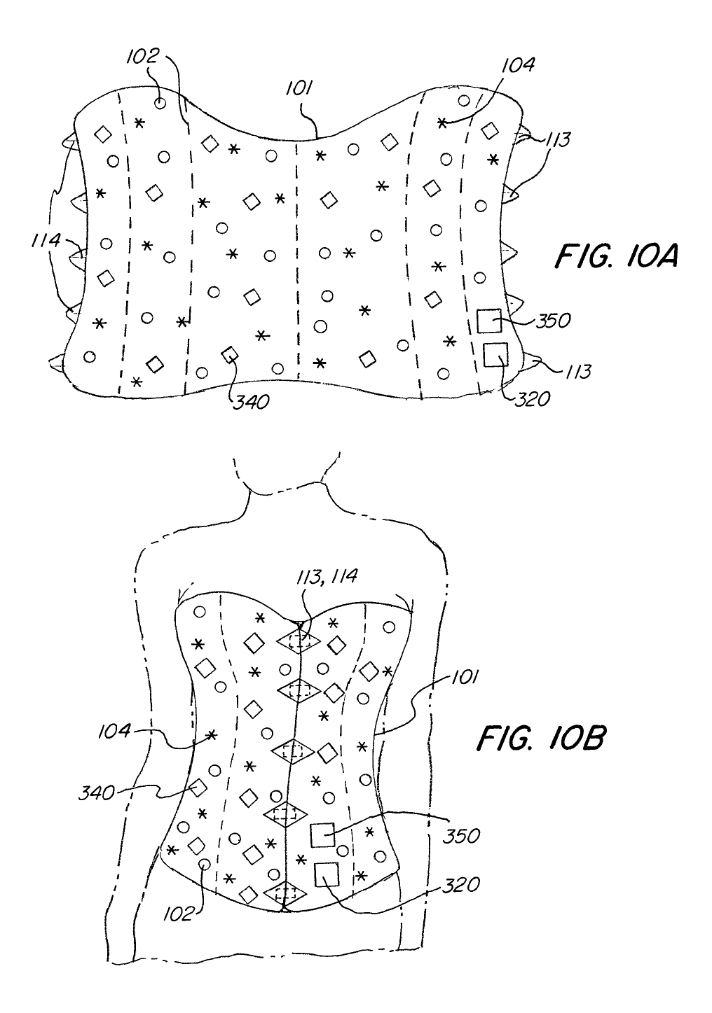

The shape memory material 102 allows the pair of clasp bands/straps 10 to transform to their original form (a more stable form) upon receiving a stimulus and cause the pair of clasp bands/straps 10 to bend and its two distal end portions 264 to move toward each other, and would wrap around an object if present, also called “self-assembly”. As shown in FIG. 10, the two end portions 264 move closer to each other, and the two clasp members 113, 114 clasp to connect the two clasp bands/straps of FIGS. 1A and 1B. Thus, the clasp bands/straps in FIGS. 1A to 10 may be called “half” bands/straps because two such bands/straps are required to form a clasp.

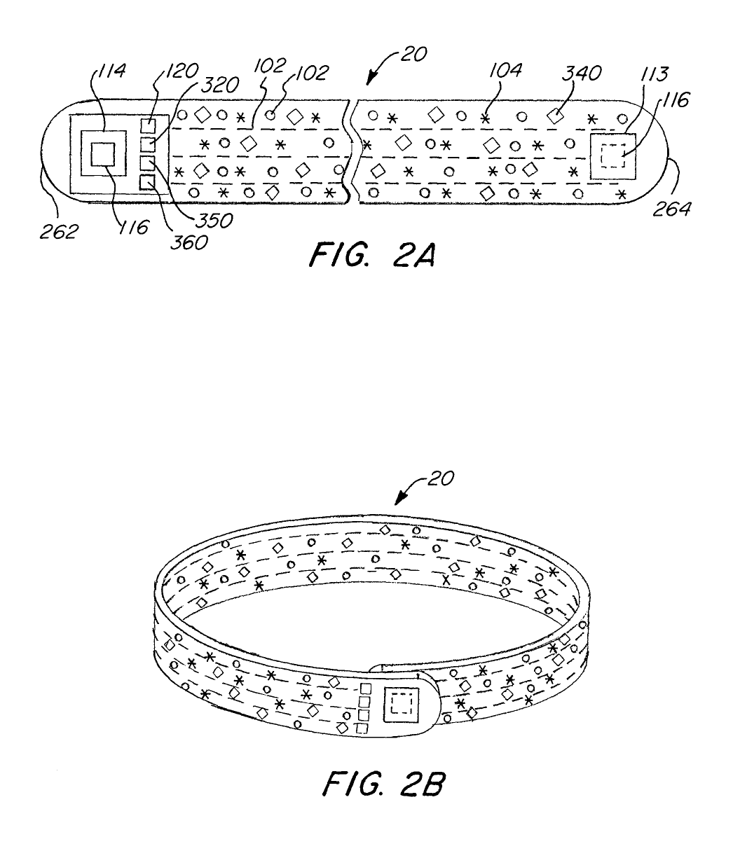

FIG. 2A shows another embodiment of the clasp bands/straps. The clasp bands/straps 20 are elongated bands/straps comprising a shape memory material 102 and a non-shape memory material 104. The clasp bands/straps 20 may further comprise a liner layer 206 on which the shape memory material 102 and the non-shape memory material 104 are deposited. The two ends 262, 264 of each of the clasp bands/straps 20 comprises two clasp members 113, 114 of a clasp. The clasp bands/straps 20 may comprise a trigger source 120 in communication with the shape memory material 102 and configured to provide a stimulus to the shape memory material 102.

Upon receiving a stimulus, the shape memory material 102 transforms from the current temporary form to its original form (a more stable form), causing the pair of clasp bands/straps 20 to deform and bring the two ends 262, 264 to move toward each other, and would wrap around an object if present. As the two end portions 262, 264 move closer to each other, the two clasp members 113, 114 clasp to form a loop. (FIG. 2B).

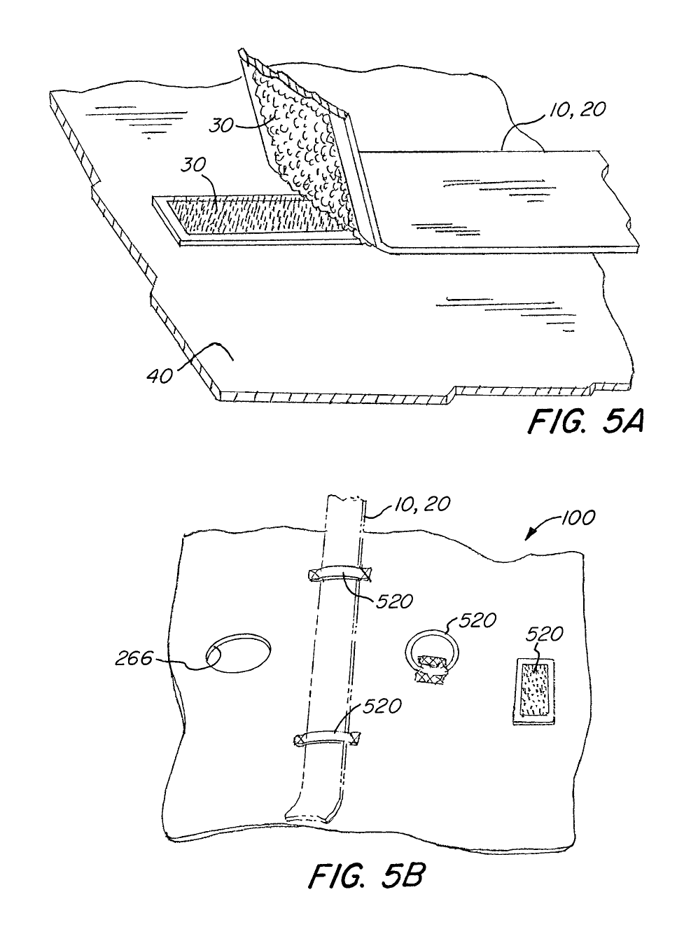

The clasp bands/straps 10, 20 have two opposite surfaces of substantially the same area and shape. In some embodiments, as shown in FIG. 5A, one surface of the clasp bands/straps 10, 20 may comprise a fastening means for connecting the clasp bands/straps 10, 20 onto a surface of another object. The fastening means may be a permanent adhesive, which will make the clasp bands/straps 10, 20 permanently adhered to the surfaces they attached. In preferred embodiments, the fastening means is a hook-and-loop fastener 30, such as a Velcro strap. When the surface of another object 40 (such as a splint, a cast, or a brace) provides a matching hook-and loop fastener, the clasp bands/straps 10, 20 easily and removably attach onto the object 40. Once attached to the object, the self-assembly triggered clasp of the clasp bands/straps 10, 20 may help the object to self-assemble, if feasible, and to close an opening of the object. One-piece elongated bands/straps 20 may close and/or support an object even without previously attaching to the subject. For example, a one-piece clasp band/strap may be placed around a splint with the two loose ends of the one-piece clasp hanging around the splint but not in contact with each other. The phase transition of the shape memory material of the clasp band/strap brings the two loose ends close to each other, thereby facilitating the clasp of the two ends of the one-piece band clasp. Upon the clasp, the one-piece band/strap forms a hoop which encircles and conforms to the shape of the splint, thereby supporting the splint and/or closing an opening of the splint.

The shape memory material 102 may be formed from of one or more shape memory polymers (SMPs), one or more shape memory alloys (SMAs), or a mixture thereof. Noticeable changes include the change of the band/strap length and the curving effect of the clasp bands/straps. When a stimulus is applied or fed to the shape memory material, the modulus of elasticity of the material can change from a rigid or semi-rigid state to a flexible, malleable state suitable for reshaping and stretching the material. In some embodiments, the stimulus comprises application of electric current. FIGS. 1A, 1B, and 2A shows a lateral cross-sectional view of the clasp bands/straps 10, 20 having a shape memory material 102 in the form of wires and particles. The SMP, SMA, mixture, composite, compound or fabric are shaped in such a manner such that they may feature distinctively shaped shape transitions, having different shape transition conditions, which may be initiated by different external factors or stimuli.

Suitable SMPs that may be used in the present invention include, but are not limited to, polyesters, polycarbonates, polyethers, polyamides, polyimides, polyacrylates, polyvinyls, polystyrenes, polyurethanes, polyethylene, polyether urethanes, polyetherimides, polymethacrylates, polyoxymethylene, poly-c-caprolactone, polydioxanone, polyisoprene, styrene copolymer, styrene-isoprene-butadiene block copolymer, cyanate ester, copolymers of stearyl acrylate and acrylic acid or methyl acrylate, norbonene or dimethaneoctahydronapthalene homopolymers or copolymers, malemide, silicones, natural rubbers, synthetic rubbers, and mixtures and compositions thereof. Further, the SMPs may be reinforced or unreinforced SMP material.

Suitable SMAs that may be used in the present invention include, but are not limited to, copper-aluminum-nickel alloys, nickel-titanium alloys, copper-zinc-aluminum alloys, iron-manganese-silicon alloys, gold-cadmium, brass, ferromagnetic, other iron-based alloys, and copper-based alloys. In a preferred embodiment, nitinol wires are used as the shape memory material. The nitinol wires, upon stimulation, will deform primarily in radius which creates both a tension and pressure type of adjustment. In one embodiment, the nitinol wires contract by about 4% to about 5% at 80° C.

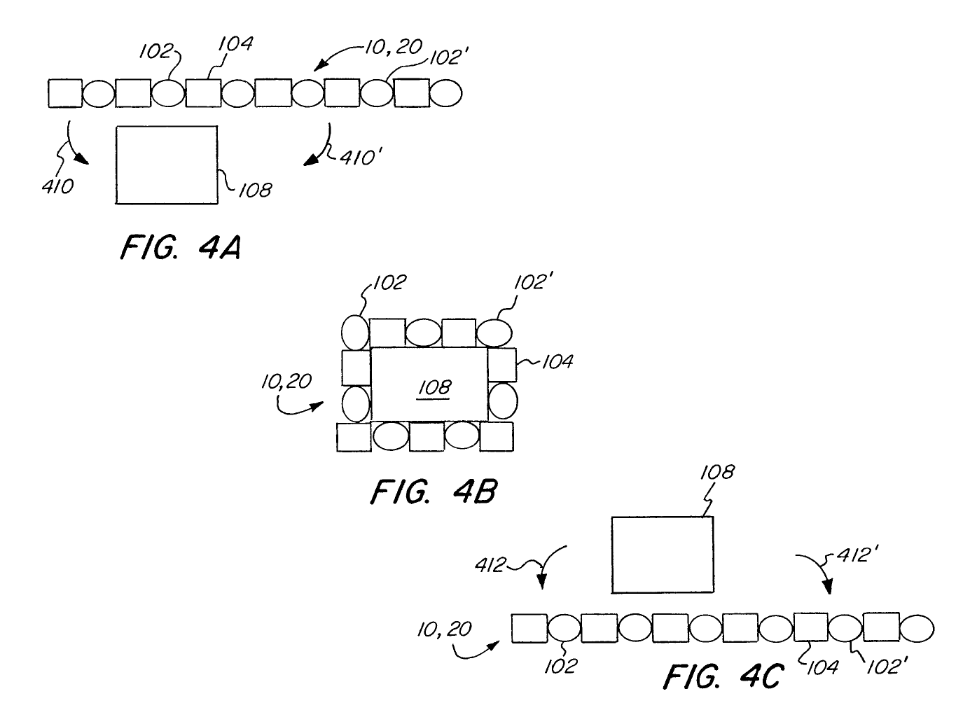

In some embodiments, the shape memory material comprises more than one shape memory material 102, 102′ that provide counteracting actuations simultaneously, in directions 410, 410′, from the memorized shape, as illustrated in FIG. 4A. The counteracting actuation function similar to muscle contraction in which the biceps and triceps provide for flexion and extension of the elbow joint, thereby contributing to functional movement of the arm. The two or more shape memory materials 102, 102′ are adapted to counteract one another so that the clasp bands/straps 10, 20 are able to self-assemble from a memorized shape (see FIG. 4A for example) to a first temporary shape (see FIG. 4B for example), cease self-assembly and maintain the first temporary shape. Additionally, the counteracting actuations of the two or more shape memory materials 102, 102′ provide for adaptive adjustment (gradualism) of the clasp bands/straps 10, 20 from the first temporary shape to other intermediate temporary shapes in order to compensate for changes in shape and/or size of the underlying object 108.

Thereafter, if a “removal” trigger is transmitted by the trigger source to the shape memory material 102, 102′, the clasp bands/straps 10, 20 may automatically disassemble in directions 412, 412′, opposite to the directions 410, 410′, respectively, thereby reverting back to its memorized shape (e.g., flat shape), as shown in FIG. 4C. The non-shape memory material 104 may comprise, but is not limited to, one or more of the following materials: plastic, metal, rubber, fabric, mesh or ceramic. The non-shape memory material 104 may provide some rigidity and structural stability to the overall arrangement of the smart material. However, the non-shape memory material 104 does not prevent the clasp bands/straps 10, 20 as a whole from transitioning between different shapes.

The liner 206 may be a form liner and/or a mesh layer. The mesh layer may comprise a plastic material or textile (e.g., fabric) material. The process of combining or intercalating the mesh layer and shape memory materials 102 and non-shape memory materials 104 may involve threading, casting, coating, welding, and/or bonding.

The clasp for use on the clasp bands/straps 10, 20 may be any type of clasp. Preferably, the clasp is a magnetic clasp. In that preferred embodiment, the clasp members 113, 114 comprise magnetic pieces 116, which may mutually attract and magnetically connect to each other to form an overlap to close the loop, without a prior physical contact. The magnetic pieces 116 may be of any suitable shapes. Since the magnetic force of attraction decreases with distance, this force is exerted most between the first and second magnet pieces when they are directly and substantially superposed on each other. Accordingly, not only should the two magnet pieces be matched magnets (namely, they are polarized in the same direction) so that they can be superposed on each other, the two magnet pieces also, preferably, have substantially the same size and same shape to maximize the exertion of magnetic force. The magnetic force between the magnet pieces causes the clasp members to adhere strongly to each other.

The magnet pieces may be permanent magnets made of neodymium-iron-boron. Those skilled in the art will understand that the mutually attracting magnetic pieces described previously could be electromagnetic fields or any other force types that can mutually attract and lock together. To provide additional magnetic shielding, the wearable band/strap may have removable or fixed magnet shields which are sufficiently large to attach and cover the outer surfaces of the band/strap. In a preferred embodiment, the shields are made of Mu shielding material.

The overlap formed by the magnetic pieces may have a tab, an indentation, or a button on an edge of the clasp members 113, 114 so that a user may easily lift up or push away one of the clasp members with a finger in order to open the engaged clasp members. A skilled artisan will understand that there are other mechanisms known in the art, such as an automatic mechanism with a remotely controlled motor, may be used to separate two attracted magnet pieces. Since the magnetic force of attraction decreases with distance, only an initial force is needed to break the attraction between the two magnet pieces. One advantage of the magnetic clasp in accordance with the present invention is that it can be easily operated (i.e., open and closed) with a single hand or hands free.

In some preferred embodiments of the invention, the clasp bands/straps 10, 20 as shown in FIGS. 1A, 1B and 2A may further comprise at least one motor 320 disposed on one of the clasp members (e.g., 113, 114) or on the clasp bands/straps 10, 20 for fine tuning the tightness of the clasp bands/straps initially and during the courses of use. The clasp bands/straps 10, 20 may further comprise sensors 340 and a control unit 350 which is in communication with the sensors 340 and the at least one motor 320. The sensors 340 may be positioned on the clasp bands/straps 10, 20 and may be remotely positioned from the clasp bands/straps. The sensors 340 are configured to acquire information related to the clasp bands/straps 10, 20 and send sensed or acquired information (e.g., measurements) to the control unit 350.

Suitable sensors may be touch sensors, pressure sensors, force sensors, capacitive sensors, conductivity sensors, light or optical sensors, heat sensors, strain gauges, stress gauges, bend sensors, magnetic sensors, location sensors, accelerometer sensors, mechanical sensors (e.g., external buttons or levels, removable tabs/rods/latches, external sliders, bending-release latches, etc.), or a combination thereof or any additional type of sensor. In some embodiments, the sensors are configured such that number, configuration, type and pattern of the sensors in contact with a limb or a splint determines timing for closing the band/strap and tensioning of the band/strap. A user may select number, configuration, type, and pattern of the sensors to be in contact with a limb or a splint and enter the selections in the user input unit so as to control timing for closing the band/strap and tensioning of the band/strap.

Based on the information received from the sensors 340, the control unit 350 may determine whether the motor 320 needs to be activated to loosen or tighten the clasp bands/straps 10, 20 and if so, the particular movement to be carried out by the motor 320 to reach the desired effect. The control unit 350 then sends triggering signals to the motors 320 to activate that movement. The movement of the motor 320 changes the relative position of the clasp 113, 114 with respect to the clasp band/strap 10, 20 thereby fine tuning the fitting of the underlying subject.

For example, if the measurements from the sensors 340 indicate that the clasp bands/straps 10, 20 are too loose, as compared to a threshold value, the control unit 350 may activate the motor 320 in order to tighten the clasp bands/straps 10, 20; conversely, if the measurements from the sensors 340 indicate that the fitting is too tight, as compared to a threshold value, the control unit 350 may activate the motor 320 in order to loosen the clasp bands/straps 10, 20. This process may also be characterized as a sensor triggered activation. When a threshold tightness level is reached after the motor movement and detected by the sensors 340, the sensors 340 will communicate with the control unit 350, which triggers the motors 320 to stop its movement. In some embodiments, the control unit 350 may be a central processing unit (CPU). In other embodiments, the control unit 350 may be a simple circuit for receiving inputs and providing an output according to the inputs to motors 320.

Additionally, the motor may be used to superimpose two matched magnet pieces on each other for maximum magnetic force. In some embodiments, the control unit is configured so that, before clasping, the control unit instructs the motor to adjust the position of the second clasp member so that the two distal ends are aligned on top of each other with a magnetic piece on each end facing each other, thereby facilitating the two magnetic pieces to clasp by magnetic force.

The control unit 350 may be disposed in many places. In some embodiments, the control unit 350 may be disposed distantly away from the clasp or the splint. In other embodiments, the control unit 350 may be disposed in the clasp bands/straps, the clasp, or the splint to which attached the clasp bands/straps. In one embodiment, the control unit 350 may be disposed in the clasp members 113, 114.

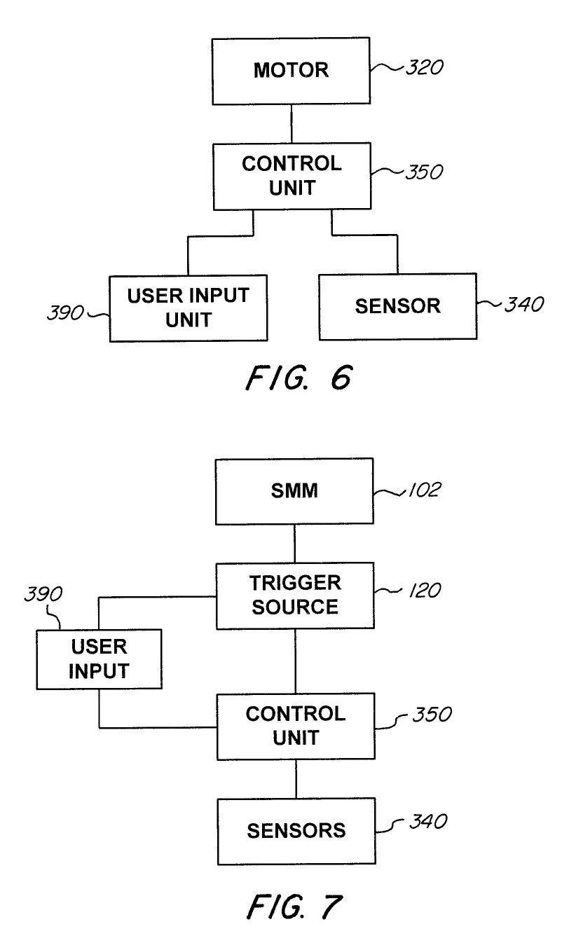

In addition to the sensor triggered activation, activation of the motor 320 may be triggered by a user input. This process may also be called a user triggered activation. FIG. 6 is a block diagram showing the two types of activation mechanisms. In this diagram, the control unit 350 communicates with the sensors 340, which may trigger activation of the motor 320 through the control unit 350. At the same time, the control unit 350 also communicates with a user input unit 390. Upon receiving a triggering signal from the user input unit 390, the control unit 350 activates the motor 320 in accordance with the user input. The user input unit 390 may be a push button that can be pushed to activate the motor 320. The user input unit 390 may also be an interface on a computer, a handheld remote control, or on a smart watch which allows a user to manually provide instructions. A user may also set or change a threshold tightness level before or during wearing of the band/strap by using the interface. The present invention advantageously allows for setting different tightness for different people as some people may not want a band/strap to be in full contact with their skin but would rather have some degree of slack in the final fit.

If the activation of the motor 320 is only triggered by the sensors 340, then the adjustment is completely automatic. The activation of the motor 320 may be triggered by the sensors 340 and a user input unit 390 consecutively. The control unit 350 is configured so that, if the control unit 350 receives information from the user input 390 and the sensors 340 simultaneously, the information from the user input unit 390 controls.

Those skilled in the art understand that the control unit contains additional controls as necessary to work the invention correctly. Examples of such control would be an alarm/notification, automatic conversion to manual control, or automatic release of the tightness of the clasp/band/strap assembly for safety purposes if the sensors determine it is tightened beyond safe parameters programmed into the control unit.

The control unit 350 may also be in communication with the trigger source 120 to control the activation and deactivation of the trigger source 120. For example, the control unit 350 may instruct the trigger source 120 to send stimulus to the shape memory material or cease stimulation based on sensed information from the sensors 340. The user input unit 390 may be configured to directly control the trigger source 120. FIG. 7 is a block diagram showing the activation mechanism.

According to instructions from the user input unit 390, the trigger source 120 may generate a stimulus to the shape memory material 102. The user input unit 390 may be in the form of, for example, a switch, a knob, a push button, or a touch screen. In one embodiment, the user input 390 is a push button located on a splint, cast, or brace. After the push button is pushed, the trigger source 120 creates and applies a stimulus (e.g., electric circuit) to the shape memory material 102, causing the shape memory material 102 to deform, and the two end portions of the pair of clasp bands/straps 10 to bend and approach one another. In other embodiments, the user input unit 390 is an interface on a computer, a handheld remote control device, or a smart watch, in which case, the trigger source 120 may receive instructions directly from the touch screen of a computer, a handheld remote control device, or a smart watch. The user input unit 390 may also allow a user to set threshold levels of various sensors. It may further allow a user to select the types and locates of various sensors dispersed on the clasp band/strap, clasp, and/or splint.

In a preferred embodiment, a remote control unit wirelessly, for example, via a blue tooth device, communicates with the shape memory alloy wires in each of the pair of clasp bands/straps. The remote control unit initiates a first of the pair of clasp bands/straps to bend with its end moving toward the center of the arc of desired motion, and subsequently initiates a second of the pair of clasp bands/straps to bend with the end moving along the same arc of motion so that the two ends are aligned on top of each other with a magnetic piece on each end facing each other before clasping, while compensating automatically for any mal-position that may occur when the clasp band is initially placed on the splint. In these embodiments, the pair of clasp bands/straps are individually constructed, each half band comprises its separate shape memory material, separate trigger source, separate sensors, etc. Laser beam detection sensor mechanisms, RF sensor mechanisms, or any other sensor mechanism may act as on/off controllers for timing the synchrony of the SMA's and SMP's closures with the timing of the magnet locking or matching mechanisms or mechanics of closure timing.

Motors suitable for use in the present invention may be any type, including, but not limited to, an electric motor, an electrostatic motor, a pneumatic motor, a hydraulic motor, a fuel powered motor. In a preferred embodiment, the motor is an electric motor that transforms electrical energy into mechanical energy. Additionally, the motor should be small enough to be housed in a clasp member. It is also preferred that the motor can complete the tensioning or fine tuning quickly upon receiving instructional triggering signals. For example, in some embodiments, it takes the motor 320 as short as 1-2 seconds to increase or decrease a relative position by approximately +/−6 mm to achieve a fine tuning. Commonly known electric motors such as a lead screw actuator, a worm-gear type motor, or a rack and pinion motor, ratcheting motor, hydraulic, pneumatic or other types of motors may be used in the present invention.

By using sensors to acquire information and trigger the activation and/or deactivation of the motor in order to fine tune the tightness of the clasp band/strap as needed, the present invention advantageously provides a clasp band/strap that not only can close by self-assembly but also can automatically adjust and substantially maintain a preferred tightness thereof during using.

The clasp band/strap 10,20 may further comprise at least one power source to supply power to the motor 320, and optionally also supply power to the control unit 350, the trigger source 120, and the sensors 340. In some embodiments, the motor 320 may be associated with an external battery 360, as shown in FIGS. 1A, 1B, and 2A. In preferred embodiments, the motor 320 may include an internal battery (not shown). An external battery may also be housed in the composite 101. The battery may be any type, shape, or form of battery. It may be a disposable battery or a rechargeable battery. The control unit contains a program to notify the user of need to replace a disposable battery or to charge the rechargeable battery.

While FIGS. 1A, 1B, and 2A show examples of a single clasp band/strap housing many components (e.g., a motor, a control unit, a battery, and sensors), a skilled artisan will understand that those components may be housed in different places. For example, a control unit and sensors may be placed externally from the clasp band/strap. Moreover, a skilled artisan will understand that the present invention also encompasses two motors and/or two controllers to provide multiple independently controlled actuations (not shown), especially for the two “half” bands/straps in FIGS. 1A and 1B.

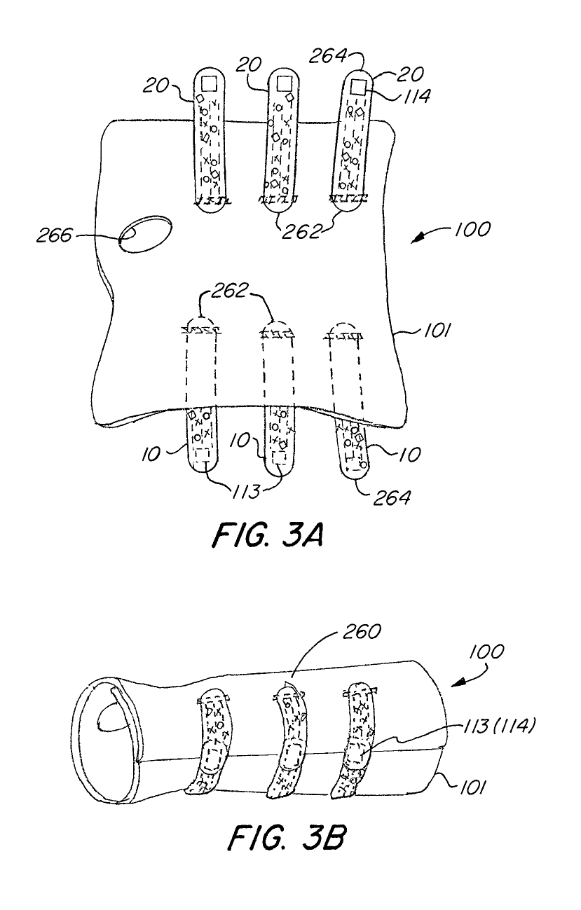

The clasp bands/straps 10, 20 are useful to immobilize and/or fix an orthopedic devices (e.g., casts, splints, braces) without using hands to maneuvering the clasp bands/straps and the devices. FIGS. 3A and 3B show an exemplary embodiment of an immobilization and fixation device 100 (e.g. braces, splints, casts) in its open and closed positions. The immobilization and fixation device 100 includes a composite 101 adapted to be placed around the body part and to provide strength and weight-bearing support to the body part in a closed, working position. In some embodiments, the composite 101 may include at least one foam layer to provide protection and comfort to the wearer. It may also comprise a fabric liner for contact with a body part. One skilled in the art, as already mentioned above, would know that it may be composed of any number of materials in laminate or other composite form that result in the correct pliability, comformability, and strength required by the device to function correctly.

The immobilization and fixation device 100 may include one or more holes or apertures 266 formed in the composite 101. The hole 266 is adapted to receive a patient's thumb, fingers, toes, or other digits, or accommodate a joint. The hole 266 may be pre-formed in the composite 101 during a manufacturing/production process. Alternatively, the composite may not be pre-formed with a hole 266, and instead, a medical practitioner can perforate the composite 101 using scissors or another cutting tool. The medical practitioner, therefore, can customize the hole 266 to the specific size, shape, and position of the patient's digits or joint.

The device 100 includes a plurality of pairs of clasp bands/straps. The elongated clasp bands/straps 10 comprise a fabric layer or other type of material layer 206 on which a shape memory material 102 and a non-shape memory material 104 are deposited. A trigger source 120 may also be provided on the clasp bands/straps 10. Each of the clasp bands/straps 10 has a proximal end 262 and a distal end 264. Two clasp members 113, 114 of a single clasp are attached to the distal ends 264 of each pair of clasp bands/straps 10 so as to connect or disconnect the pair of clasp bands/straps 10. By using the “half” bands/straps, the number of clasps is half of the number of the clasp bands/straps.

Though in FIGS. 3A and 3B, only the “half” clasp bands/straps are shown, a skilled artisan would understand that the one piece clasp band/strap 20 would work equally well under the same concept. By using one-piece bands/straps in which both ends receive one clasp member (two clasp members per clasp), the number of clasps needed in the device is the same as the number of clasp bands/straps.

The clasp bands/straps 10 may be permanently attached to the device 100 by being sewn or otherwise permanently bonded to the device. For example, the proximal end 262 of the clasp bands/straps 10 may be sewn to the fabric liner of the composite 101 and thus permanently attached to the device 100. Alternatively, the clasp bands/straps 10 may be removably attached to the device 100 by attaching to anchors 520 on the device, as shown in FIG. 5B. The anchors 520 may be buckles, Velcro strap, or other adhesives. The clasp bands/straps may comprise adhesive material on their backs for attaching to the anchors 520 or simply tying or threading through the anchors 520. In a preferred embodiment, the clasp bands/straps have a Velcro strap on their back which can be easily and removably attached onto the corresponding Velcro strap on the device. The functional length of the clasp bands/straps can be adjusted based on the extent of the overlap between the Velcro piece on the device and the Velcro piece on the clasp bands/straps, and the positions where the Velcro pieces are placed on the device. Thus, the present invention provides a convenient means to adjust the length of the clasp bands/straps, and consequently, the tightness of the composition when it is in a working position.

The self-closing and self-adjusting splint device can be easily prepared by starting with a commercially available splint. For example, a doctor or an orthotic shop may take a commercial wrist splint off the shelf, place it loosely on a patient as is for sizing and configuration. The commercial splint comes with an attached Velcro hook and loop fastener which has to be pulled by a patient or a fitting person (a doctor or an orthotist) through a buckle and then closed—the conventional method to close and tighten a split. However, instead of using the Velcro hook and loop fastener in the conventional way, a doctor or an orthotist may cut the Velcro hook and loop strap. The shorten Velcro piece which is still attached to the splint is then attached to a mating Velcro piece attached to a half clasp band/strap of the present invention. The mating Velcro may be attached to the half clasp band/strap by glue, adhesive strip, or sewing in to bind those two. On the side of splint where the buckle is positioned, a similar strap of Velcro is added to the surface of the splint with an adhesive (or sewn in or however on either side of the splint). After that, the added Velcro strap on the splint is attached to another mating Velcro with a half clasp band/strap. The two half clasp bands/straps are positioned on the splint in a way that they would clasp when the splint-clasp band/strap device deforms upon receiving a stimulus. As explained before, the functional length of the clasp bands/straps can be easily adjusted by controlling the extent of overlap between the Velcro piece on the splint and the Velcro piece on the half clasp bands/straps. Thus, the splint-clasp band/strap device is particularly suitable for customized fitting.

In addition to using a commercially available splint and attaching it with clasp bands/straps having a shape memory material to construct a self-closing and self-adjusting splint device of the present invention, one of ordinary skill in the art would understand that one can also start with a splint already embedded with a shape memory material to prepare a self-closing and self-adjusting splint device.

The clasp for use on the clasp band/strap may be any type of clasp. Preferably, the clasp is a magnetic clasp. The device 100 may further comprise a motor actuation for fine tuning of the fitting of the device. The motor, the sensors, the control unit, the magnetic clasp, a power source, and the clasp band/strap) of the device 100 are similar to those of the clasp bands/straps 10, 20, the motor, the sensors, and the control unit of the device 100 may be disposed on the other components of the device 100. The motor 320 may be controlled by a user input unit 390 and by a control unit 350 based on sensed information, as described in FIG. 6. Most other information about the motor, the sensors, the control unit, the magnetic clasp, the power source, and the clasp band/strap of the device 100 are similar to those of the clasp bands/straps 10, 20. The differences are that the sensors 340 may now be disposed on the inner layer of the composite 101 for measurement and the motor 320 may be placed in the composite to directly adjust its tightness. Additionally, more than one motor and more than one controller may be used for individual control of the composite and the clasp bands/straps.

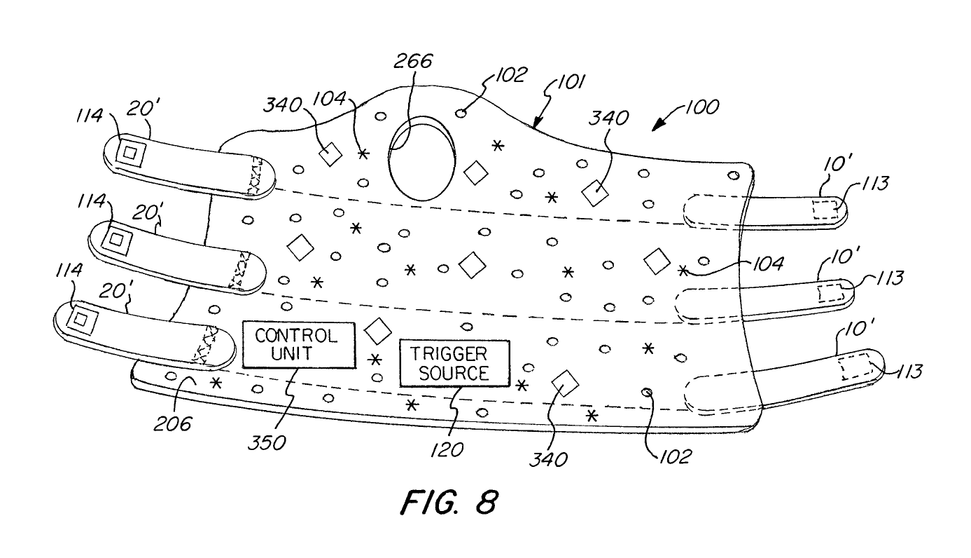

FIG. 8 illustrates another exemplary embodiment of an immobilization and fixation device 100 in which the composite 101 also comprises a shape memory material 102 and a non-shape memory material 104. The composite 101 further comprises a form liner and/or a mesh layer 206 on which the shape memory material and the non-shape memory material are deposited. The mesh layer 206 may comprise a plastic material or textile (e.g., fabric) material. The process of combining or intercalating the mesh layer 206 and shape memory materials 102 and non-shape memory materials 104 may involve threading, casting, coating, welding, and/or bonding. The device 100 may also comprise a trigger source 120 in communication with the shape memory material 102 and configured to provide a stimulus to the shape memory material 102. The composite 101 is configured to transition between a memorized (e.g., permanent) shape and multiple temporary shapes upon receiving a stimulus from the trigger source 120 upon receipt of a stimulus, wherein the composite 101 is configured to self-assemble into a first temporary shape around an appendage or body part in response to a first trigger from the trigger source 120 and to stop self-assembly in response to a second trigger from the trigger source 120. The composite 101 assembled into the first temporary shape provides strength and weight-bearing support to the body part. The device 100 is configured so that the composite 101 exerts a pressure on the body part and provides adaptive adjustment in shape in order to compensate for changes in shape and/or size of the body part and maintain the pressure substantially constant.

In some embodiments, the clasp bands/straps 10′, 20′ in FIG. 8 may simply include clasp members 113, 114 without having any shape memory materials therein. The phase transition of the composite would bring pairs of clasp bands/straps 10′, 20′ closer to each other to facilitate the clasp of the clasp members 113, 114. In other embodiments, the clasp bands/straps 10′, 20′ may have independent set of shape memory materials. In preferred embodiments, the device 100 in FIG. 8 may also further include motor actuation for fine tunings of tightness with the assistance of a control unit, sensors, and a user input, as described before. Detailed information of the device 100 in those embodiments will not be repeated here.

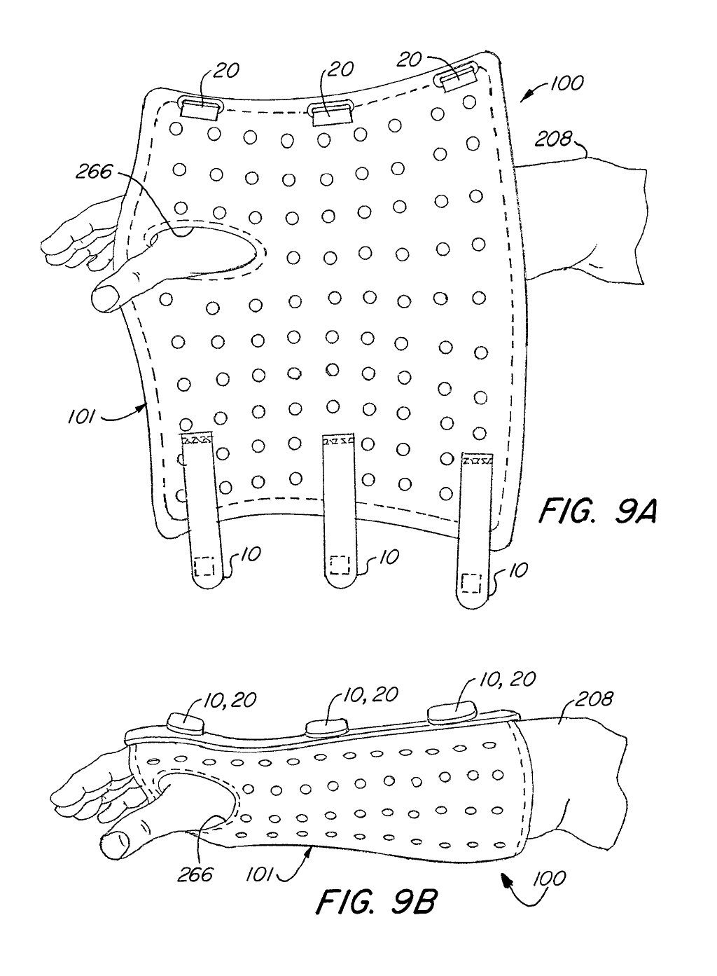

FIGS. 9A and 9B shows a process of applying the immobilization and fixation device 100 for treating a body part of a patient. When in use, a limb 208 passes through the hole 266, and the device 100 is loosely surrounded the limb. A stimulation is provided which triggers the shape memory material to change to different form, causing the clasp members to clasp, and the composite conforms to the contour of the limb. Motorized actuation is further controlled by a control unit based on a user input or sensed information.

Comparing to the traditional method to secure a splint, i.e., by threading Velcro straps through a loop and then fastening them down by hands, the device of present invention provides a novel method and device for automatically closing the splint. The clasp bands/straps not only enable self-assembly but also reduce the likelihood that the composite of the splint accidentally shifts or is removed from the body part. With the motor actuation, the clasp bands/straps may further adjust the fitting during the entire orthopedics treatment. This is particularly useful because a limb underlying the shape memory material may slightly expand and/or contract as a result of the healing process (e.g., swelling dissipates, bone alignment improves) or activities.

Another advantages of the present invention is that the clasp bands/straps can be independent items from splints. Moreover, the clasp bands/straps may be removably attached to splints, which make them flexible and amenable to repeated uses. It costs less to manufacture such clasp bands/straps than to manufacture casts, braces, or splints with pre-fixed, embedded shape memory materials. Finally, the clasp bands/straps can be of any shape and their length relative to a splint space (“the functional length”) may be easily adjusted as needed.

In another aspect, the present invention provides a tight fitting garment to be worn on a body part, such as a corset. FIGS. 10A to 10B show that such garment or item may comprise: a composite 101 having a shape memory material 102 and a non-shape memory material 104, at least one pair of clasp members 113, 114 attached to the composite, and a trigger source 120 in communication with the shape memory material. The trigger source is configured to provide a stimulus to the shape memory material. The composite is configured to transition between a memorized shape and multiple temporary shapes upon receipt of a stimulus. The composite is configured to self-assemble from a memorized shape into a temporary shape around the body part in response to a first trigger from the trigger source and to stop self-assembly in response to a second trigger from the trigger source, wherein the composite assembled into a temporary shape is adapted to affix around the body part, and the clasp members 113, 114 are clasp after the initial phase transition of the composite.

Furthermore, the garment may include sensors 340 disposed on its inner layer 206, a motor 320 disposed in one of the first and second clasp members 113, 114 and configured to adjust a position of the clasp members with respect to the composite 101, and a control unit 350 communicatively connected to the trigger source, a motor, and sensors for adjustment during wearing. The control unit regulates an amount of pressure exerted by the composite on the body part detected by the sensors by control the activation of the motor.

In some embodiments, the garment is an article of clothing composed of a regular, non-shape memory material only. The garment is equipped with at least one pair of clasp bands/straps attached to the article for closing the garment. Each clasp band has one end attached to the article and the other end comprising a clasp member, wherein the clasp members on each pair of clasp members are configured to clasp so as to close the garment. Each clasp band/strap also comprises a smart material (e.g. a shape memory material) and a trigger source in communication with the shape memory material. The trigger source is configured to provide a stimulus to the shape memory material. The clasp bands/straps are configured to transition between a memorized shape and multiple temporary shapes upon receipt of a stimulus. The phase transition of the clasp bands/straps will bring each pair of the clasp bans closer to each other, thereby pulling the article around a body part (the so called “self-assembly”). Eventually, the phase transition will lead the clasp members on the clasp bands/straps to clasp, thereby affixing the article around the body part. Thus, the clasp bands/straps of the present invention may be used to substitute for the buttons of a standard garment (e.g., a shirt). A user may put on such garment without the need to button buttons with their fingers.

The garment may further include sensors disposed on its inner layer or on the clasp band/strap, a motor configured to adjust the tightness of the clasp band/strap, and a control unit communicatively connected to the trigger source, a motor, and sensors for adjustment during wearing, as described in the embodiment that garment itself is an article of clothing comprising a shape member material.

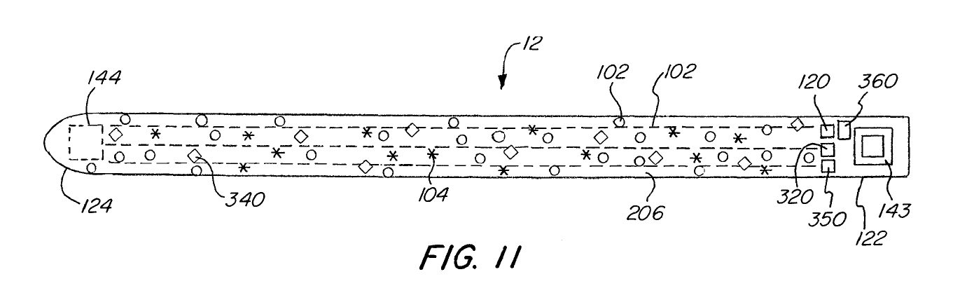

In a further aspect, the present invention provides a self-closing and self-adjusting belt which can be worn with pants around a waist of a person in lieu of a conventional loop-and-notch belt. As illustrated in FIG. 11, the belt 12 has a flexible elongated body having two ends 122, 124. The belt 12 may be made of, for example, leather, faux leather, plastic, nylon, metal, or a mixture thereof. It may be composed of a single elongated solid piece (e.g., a leather strap) or multiple solid links (e.g., linked metal rings). The belt 12 may comprise a shape memory material 102. The belt 12 may also comprise a trigger source 120 in communication with the shape memory material 102 and configured to provide a stimulus to the shape memory material 102. The shape memory material 102 is configured to change shapes between a permanent phase to a temporary phase upon receiving a stimulus.

The belt 12 may further comprise a buckle 14 having two buckle pieces 143, 144. The two buckle pieces 143, 144 are attached to the two ends 122, 124 of the belt 12, respectively, by anchoring, bolting, or other conventional means. The two buckle pieces 143, 144 may clasp to each other, just like the pair of clasp members 113, 114 would, as discussed in the earlier embodiments. The clasp and the separation of the buckle pieces 143, 144 connects and disconnects the two ends 122, 124 of the belt 12, respectively.

Upon receiving a stimulus from a trigger source 120, the shape memory material 102 transforms to its original form (a more stable form), which in turn, causes the belt 12 to curve and its two end portions 122, 124 to move toward each other—so called “self-assembly”. As the two end portions 122, 124 move closer to each other, the two buckle pieces 143, 144 clasp to connect the two end portions 122, 124, which forms a loop of the belt 12. As such, the belt 12 is able to self-close, hands-free. In a preferred embodiment, the belt, upon self-closing, conforms to the waist of the wearer.

The belt 12 may further include sensors 340 disposed on its inner layer 206 of the belt 12 in contact with pants, a motor 320 disposed in one of the buckle members 143, 144 and configured to adjust a position of the buckle members with respect to the belt 12 so as to adjust the overall length of the belt, and a control unit 350 communicatively connected to the trigger source 120, a motor 320, and sensors 340 for adjustment of tightness during wearing by control the activation and deactivation of the motor based on detected information by the sensors. As a result, the belt 12 of the present invention is able to perform fine tensioning of the belt upon initial closing and during wearing as needed, without use of hand to physically touch and maneuver the belt.

In one embodiment, one of the buckle members may further house a battery (i.e., a power source) for supplying power to the trigger source, the motor, the control unit, etc. as shown in FIG. 11. In another embodiments, a battery, a control unit, and a motor, etc. are centrally located and communicate to the clasps at either end of the assembly. In a further embodiment, the trigger source, the battery, and the control unit may be positioned in the belt or remotely from the belt and the buckle.

The types of buckle pieces 143, 144 may be substantially the same as the clasp members 113, 114. The trigger source 120, the shape memory material 102, the motor 320, the sensors 340, the control unit 350, and the battery 360 may be identical to or substantially the same as those described in the other embodiments of the invention. In a preferred embodiment, the buckle 14 comprises a magnetic clasp, and the shape memory material is nitinol. The belt 12 may be so designed that it will enable a sequentially curving of the two ends of the belt (i.e., one end curves first and the other end curves second), followed by aligning the magnetic clasp pieces to effectuate clasping, just like in the clasp band/strap 10, 20 embodiments disclosed earlier. Finally, as discussed in the earlier embodiments, the trigger source, the motor, and the control unit can be controlled by a user's input. Detailed information of these components and their respective functions in the belt embodiment will not be repeated.

Although the clasp bands/straps and a device comprising the clasp bands/straps according the present teachings has been shown to have applications in the medical immobilization and fixation field and in wearable technology, the clasp bands/straps can have application in various other industries, such as biomedical devices and robotics.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings.

What is claimed is:

1. An immobilization and fixation device for treating a body part of a patient, comprising:
a composite adapted to perform a therapeutic function;
a plurality of clasp bands attached to the composite, each of the plurality of clasp bands having a proximal end and a distal end,
a plurality of clasps, each of the plurality of clasps having two clasp members,
wherein each of the plurality of clasp bands attaches to one or two of the clasp members on one or both of its proximal and distal ends,
a shape memory material disposed in the plurality of clasp bands,
a trigger source in communication with the shape memory material,
wherein the trigger source is configured to provide a stimulus to the shape memory material,
wherein the shape memory material is configured to transition between a temporary shape and a memorized shape automatically upon receipt of a stimulus, and
wherein the transition of the shape memory material causes the two ends of the plurality of clasp bands to move towards to each other, thereby facilitating the clasp of the two clasp members.

2. The device of claim 1,
wherein each of the plurality of clasp bands attaches to one of the clasp members.

3. The device of claim 1,
wherein each of the plurality of clasp bands attaches to two clasp members on its proximal and distal ends respectively.

4. The device of claim 1, wherein the plurality of clasp bands are removably attached to the composite.

5. The device of claim 1,
wherein the shape memory material comprises two shape memory materials,
wherein the two shape memory materials provide counteracting actuation such that a first shape memory material is configured to shape transition in a first direction in response to a first stimulus and a second shape memory material is configured to shape transition in a second direction in response to a second stimulus simultaneously, the second direction being opposite the first direction.

6. The device of claim 1, wherein at least one of the plurality of clasps is a magnetic clasp, wherein the two magnetic pieces of the magnetic clasp are mutually attracted to each other by magnetic force over a space, such that the clasp members clasp to form an overlap without prior physical contact.

7. The device of claim 1, further comprising:
a motor disposed in a first clasp member, the motor being configured to adjust a position of the plurality of clasps with respect to the plurality of clasp bands in order to tighten or loosen the plurality of clasp bands,
sensors disposed on interior surfaces of the plurality of clasp bands, the composite, and a combination thereof, and
a control unit in communication with the motor, the sensors, and the trigger source,
wherein the control unit is configured to instruct the trigger source to send a stimulus to the shape memory material; and
wherein the control unit is configured to control activation of the motor based on measurements provided by the sensors.

8. The device of claim 7,
wherein the control unit is configured to start the activation of the motor if the measurements provided by the sensors are higher or lower than a predetermined threshold value, and
wherein the control unit is configured to cease the activation of the motor if the measurements provided by the sensors reach the predetermined threshold value.

9. The device of claim 7,
further comprising a user input unit in communication with the trigger source and with the control unit,
wherein the control unit is configured to control activation of the motor in response to instructions provided by the user input unit, and
wherein the trigger source is configured to send a stimulus to the shape memory material in response to instructions provided by a user input unit.

10. A clasp band for facilitating an automatic closure comprising:
a first half band having a proximal end and a distal end,
a second half band having a proximal end and a distal end,
the first and second half bands being configured to be removably attached to an limb at least at the proximate ends of the half bands,
a clasp having first and second clasp members attached to the two distal ends of the first and second half bands respectively so as to connect or disconnect the first and second half bands at the distal ends,
a first shape memory material disposed in the first half band,
a second shape memory material disposed in the second half band,
a first trigger source in communication with the first shape memory material,
a second trigger source in communication with the second shape memory material,
sensors disposed on the first and second half bands,
a control unit in communication with the first and second trigger sources and with sensors,
wherein each of the first and second trigger sources are configured to provide a stimulus to each of the first and second shape memory materials,
wherein each of the first and shape memory materials is configured to transition between a memorized shape and a temporary shape upon receipt of a stimulus,
wherein the control unit is configured to instruct the first trigger source to provide a stimulus to the first shape memory material in response to sensed information provided by the sensors, causing the first half band to curve with its distal end moving toward the center of an arc of a closed position of the band, and
wherein the control unit subsequently instructs the second trigger source to provide a stimulus to the second shape memory material, causing the second half band to curve its distal end and move toward the center of the arc of the closed position of the band, thereby facilitating the clasp of the first and second clasp members.

11. The clasp band of claim 10, wherein the back of the two half bands comprises a hook and loop fastener.

12. The clasp band of claim 10,
wherein the first clasp member having a first magnet piece, and
wherein the second clasp member having a second magnet piece.

13. The clasp band of claim 12, further comprising a motor disposed in one of the first and second clasp members,
wherein the motor is configured to adjust a position of the clasp members with respect to the half bands,
wherein the control unit in communication with the motor,
wherein the control unit is configured to control activation of the motor based on measurements provided by the sensors.

14. The clasp band of claim 13,
wherein the control unit is further configured that, before clasping, the control unit instructs the motor to adjust the position of the second clasp member so that the two distal ends are aligned on top of each other with a magnetic piece on each end facing each other, thereby facilitating the two magnetic pieces to clasp by magnetic force.

15. The clasp band of claim 10,
wherein the sensors are touch sensors, pressure sensors, force sensors, capacitive sensors, conductivity sensors, light or optical sensors, heat sensors, strain gauges, stress gauges, bend sensors, magnetic sensors, location sensors, accelerometer sensors, mechanical sensors, or a combination thereof.

16. The clasp band of claim 15,
wherein the sensors are configured such that number, configuration, type and pattern of the sensors in contact with an limb determines timing for closing and tensioning of the first and second half bands; and
wherein a user selects number, configuration, type, and pattern of the sensors to be in contact with an limb and enters the selections in the user input unit so as to control timing for closing and tensioning of the first and second half bands.

17. The clasp band of claim 10, wherein the stimulus is application of electric current.

18. The clasp band of claim 10, wherein the proximate ends of the two half bands are connected to each other, such that the clasp band is a one-piece band.

19. The clasp band of claim 10, wherein the shape memory material is nitinol.

20. An item that is adapted to be worn on a body part, comprising:
a composite having a shape memory material and a non-shape memory material;
at least one pair of clasp members attached to the composite;
a motor disposed in one of the first and second clasp members, the motor being configured to adjust a position of the clasp members with respect to the composite,
a trigger source in communication with the shape memory material, the trigger source being configured to provide a stimulus to the shape memory material;
the composite being configured to transition between a memorized shape and a temporary shape upon receipt of a stimulus;
sensors disposed on the inner layer of the item,
a control unit communicatively connected to the trigger source, the motor, and the sensors;
wherein the composite is configured to self-assemble between a memorized shape and a temporary shape around the body part in response to a first trigger from the trigger source and to stop self-assembly in response to a second trigger from the trigger source,
wherein the at least one pair of clasp members clasps upon self-assembly,
wherein the composite assembled into the temporary shape is adapted to affix around the body part, and e
wherein the control unit regulates an amount of pressure exerted by the composite on the body part detected by the sensors by control the activation of the motor.

21. A belt with an automatic closure and self-adjusting ability comprising:
an elongated body having two end portions,
a buckle having first and second buckle pieces attached to the two end portions of the elongated body respectively so as to connect or disconnect the two end portions of the belt,
a motor disposed in one of the buckle pieces,
a shape memory material disposed in the elongated body, the shape memory material comprising a nitinol,
a trigger source in communication with the shape memory material,
sensors disposed on the elongated body,
a control unit in communication with the trigger source, the motor, and sensors,
wherein the trigger source is configured to provide a stimulus to the shape memory material,
wherein the shape memory material is configured to transition between a memorized shape and a temporary shape upon receipt of a stimulus,
wherein the motor is configured to adjust a position of one of the buckle pieces with respect to the elongated body,
wherein the control unit is configured to instruct the trigger source to provide a stimulus to the shape memory material in response to sensed information provided by the sensors, causing the band to curve with its end portions moving toward the center of an arc of a closed position of the band, thereby facilitating the clasp of the buckle pieces, and
wherein the control unit is configured to control activation and deactivation of the motor based on measurements provided by the sensors.
